(19) United States
(12) Patent Application Publication
(10) Pub. No.: US 2016/0267240 A1
(43) Pub. Date: Sep. 15, 2016

(54) SYSTEMS AND METHODS FOR AUTOMATIC REPORTING OF POINT-OF-CARE (POC) TEST RESULTS

(71) Applicant: Polymer Technology Systems, Inc., Indianapolis, IN (US)

(72) Inventor: Schuyler Buck, Indianapolis, IN (US)

(21) Appl. No.: 15/061,845

(22) Filed: Mar. 4, 2016

Related U.S. Application Data
(60) Provisional application No. 62/132,863, filed on Mar. 13, 2015.

(51) Int. Cl. G06F 19/00; G01N 33/487; H04W 4/14; G06Q 30/02; G08B 21/02; G01N 33/49; G01N 21/84

(52) U.S. Cl. CPC G06F 19/3418; G01N 33/49; G01N 33/48792; G01N 21/84; G06Q 30/0269; G08B 21/02; H04W 4/14

(57) ABSTRACT

A system for measuring and reporting a level of an analyte includes a test strip having test strip layers configured to receive a blood sample from a user, flow the blood sample through the test strip layer, and produce a detectable change in the test strip. The system further includes a meter or analyzer configured to read the detectable change in the test strip, the meter including an SMS message transmitter, the meter including a microprocessor including and executing software for converting a property read from the test strip into an analyte level and communicating that analyte level via SMS to a remote server.

### CROSS-REFERENCES TO RELATED APPLICATIONS

This Application claims the benefit of Provisional Application No. 62/132,863 filed on Mar. 13, 2015, titled “Systems And Methods For Automatic Reporting Of Point-Of-Care (POC) Test Results,” the entire disclosure of which is hereby incorporated by reference.

### BACKGROUND

Point-Of-Care (“POC”) and home testing for various blood analytes and other detectable metrics in bodily fluids is desirable for patients and doctors. In many scenarios, patients and doctors can determine critical features related to the immediate and long-term health of patients with a simple test strip used with meters or other POC analysis devices. These devices are usually simple to use and provide results within minutes. The capture and transmission of results from such POC testing is desirable and necessary in many scenarios. Typically, however, users will have to connect their devices to a computer using a physical connection such as a cord or connect the meter used in such POC testing via Wi-Fi or other mobile network. Such connection may be tedious and unreliable, requiring the user to understand significant aspects of Wi-Fi authentication and/or software. At the same time, the actual amount of data that must be transmitted is small and may include only patient identifying information, basic statistics about the user, and the test results in the form of one or more numbers.

### BRIEF SUMMARY

In one embodiment, a system for measuring and reporting a level of an analyte includes a test strip having test strip layers configured to receive a blood sample from a user and produce a detectable change in the test strip. The system further includes a meter or analyzer configured to read the detectable change in the test strip, the meter or analyzer including a short range messaging system (SMS) message transmitter, the meter or analyzer including a microprocessor including and executing software for converting a property read from the test strip into an analyte level and communicating that analyte level via SMS to a remote server. Optionally, the meter or analyzer is an optical meter or analyzer.

In another embodiment, a system for measuring and reporting a level of an analyte includes a test strip having test strip layers configured to receive a blood sample from a user and produce a detectable change in the test strip. The system further includes a meter or analyzer configured to read the detectable change in the test strip, the meter or analyzer including a short range messaging system (SMS) message transmitter, the meter or analyzer including a microprocessor including and executing software for converting a property read from the test strip into an analyte level and communicating that analyte level via SMS. The system further includes a remote server including and executing software for receiving an SMS message including the analyte level. Optionally, the meter or analyzer is an optical meter or analyzer. Alternatively, the remote server includes an SMS gateway. In one alternative, the remote server includes and executes software for storing the analyte level and correlating an indicator associated with the SMS message using a lookup table to determine the user associated with the analyte level. In another alternative, the remote server automatically sends an electronic message to the user when the user is to test for an analyte. Optionally, the central server automatically sends a message to medical personnel when the meter or analyzer reports an analyte level to the central server. In one configuration, the remote server provides a portal for the user to review historical test results. In another configuration, the remote server provides targeted information to the user through the portal. Optionally, the targeted information includes advertisements for drugs based on the user's analyte level reported from the meter or analyzer. Alternatively, the targeted information includes health management information based on the user's analyte level reported from the meter or analyzer. In another configuration, medical personnel have access to modify the targeted information received by the user. Alternatively, medical personnel have access to modify the testing regimen executed by the user, and the remote server changes its notification schedule to the user accordingly. Optionally, when medical personnel change the testing regimen for the user, testing supplies are automatically ordered for the user. Alternatively, the analyte level is automatically correlated against previous analyte levels by the remote server, and an indication is provided to the user if the analyte level is anomalous. In another alternative, the analyte level is automatically correlated against previous analyte levels by the remote server, and an indication is provided to medical personnel if the analyte level is anomalous. Alternatively, the indication is an SMS message. Alternatively, the indication is an email.

### BRIEF DESCRIPTION OF THE DRAWINGS

FIG. 1 shows one embodiment of a system for measuring and reporting analyte levels via a short range messaging system (SMS).

### DETAILED DESCRIPTION

Certain terminology is used herein for convenience only and is not to be taken as a limitation on the embodiments of the systems and methods for automatic reporting of point-of-care (POC) test results.

In one embodiment, systems and methods for transmitting medical data over a short range messaging system (SMS) include a medical device including a microprocessor and an SMS transmission chip. In operation, the medical device detects a characteristic of a patient and sends that detected characteristic of the patient over SMS which is eventually routed to a server where the result can be further cataloged, stored, matched with other patient identifying information, and routed to relevant medical professionals or other individuals.

In some embodiments, the medical device may be a meter or analyzer that receives a test strip to which a blood sample from a patient has been applied. The meter or analyzer may be an optical meter or analyzer, and the test strip in various configurations may test for blood analytes such as cholesterol, HDL, LDL, triglycerides, glucose, A1C, or a variety of other analytes found in blood.

In many alternatives, it may be desirable to protect patient identity by not providing patient identifying information in the SMS message. Instead, a server may receive the SMS message and may determine the identity of the patient based on an identifier related to the SMS message. In one example, the identifier may be a code associated with the user. At a server receiving the SMS message, the code may be correlated through a lookup table in order to provide the user identity. Information from a meter or analyzer may be provided via one or more SMS messages.

One significant advantage that this system offers over alternatives is immediacy, and it requires no user setup. SMS systems are automatically deployed in most populated areas, and SMS networks are some of the most widely available networks. SMS systems typically have very few dead spots, which increases the reliability that a message will be sent.

Advantages include: 1) Simple setup for communication from a person testing on an over-the-counter (OTC) point-of-care (POC) device to a cloud based system that will allow for sharing of critical data and information; 2) Minimize setup to use connectivity; 3) Reduce/eliminate point-in-time connection intervention by the user (e.g., connection lost and setup/connection needed again); 4) Minimize “dead” spots; and 5) Minimize interferences.

Essentially, the system includes a wireless data transmission system for SMS messages. The SMS messages are automatically addressed to a receiving server. The receiving server may include an SMS gateway.

Embodiments of the system provide for a comprehensive ecosystem to allow patients at home to quickly and easily test their glucose, cholesterol, or other applicable blood analyte, share information with their doctor, and determine and execute a proper therapeutic path. This solution will help continue to deliver on the brand promise by making testing easy and quick for the patient and seamless and efficient for the healthcare professional. Embodiments may include an analyzer and test strip system capable of automatically sending data directly to a cloud based system. From the cloud, the healthcare team will have access to an online health portal where they can review results, see trends, and decide on the appropriate therapeutic path. The healthcare professional can choose to receive notifications for every new data result, daily summaries, or notifications only for results out-of-range sent either to a mobile phone or an email address. When it is time to test again, patients can have reminders sent directly to their phone or email address. This easy customer experience is all enabled by a highly accurate test strip system and made more convenient by simple testing kits that include everything needed to complete a test.

Embodiments of the central server provide for data management, communication and portals for various entities, and data analysis for trend determination. Although in many embodiments devices communicate directly with the central server without significant human intervention, in other embodiments, data may be manually entered and recorded at the central server or uploaded in batch.

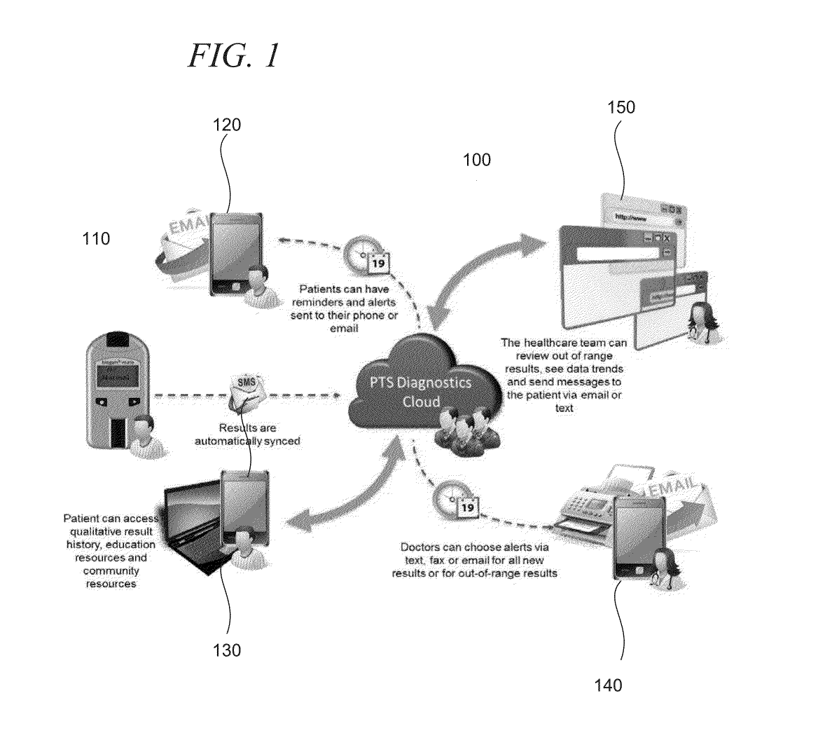

Embodiments of the system include a central server. The central server coordinates a variety of activities related to blood analyte or other testing regimens. In FIG. 1, one function that central server 100 performs is the reception of data from meter or analyzer 110. Meter or analyzer 110 may include an SMS communication system. Central server 100 may include an SMS gateway. In this use case, a user may test for an analyte. The test results may be communicated automatically via an SMS message to central server 100. The SMS message may include test results and a coded identifier for the patient. The central server 100 may correlate the coded identifier with the actual user using a lookup table. The central server 100 then may store the test results. This may occur numerous times over a period of time. In some embodiments, if the meter or analyzer cannot achieve a successfully sent SMS message, then the meter or analyzer may automatically save the test results, including a date of testing, and may retry sending the results on various schedules. Schedules include immediately at a fixed time interval until transmission is complete (e.g., once every hour), after a fixed period of time (a day has passed), after the next powerup, or various other schedules. In some alternative embodiments, the meter or analyzer may include a Bluetooth® link to a smart phone that then sends an SMS message or other transmission.

Central server 100 additionally may store information about the user, including their testing schedule. Using this testing schedule, central server 100 may provide notifications as to when the user should test for various analytes. These notifications may be SMS messages, emails, automated phone calls, calendar reminders, electronic reminders that may execute through a specialized smart phone application, or other types of electronic reminders. These notifications may be received by various electronic devices 120 of the user as applicable, including smart phones, tablets, and computers.

Central server 100 also may provide access to the user via electronic devices, including smart phones, tablets, and computers, to recorded test results, test results trends, and health information. Electronic devices 130, therefore, may communicate with central server 100. Central server 100 may automatically update for particular users depending on the results of analyte or other test results that the central server 100 receives from the user as above. The updating may include targeted advertisements for health conditions that are indicated by the user's test results. For instance, if the cholesterol of a user is high, targeted advertisements for statins or other cholesterol lowering drugs may be provided. Dietary, exercise, or other information also may be provided. In some configurations, central server 100 may indicate that an anomalous test has occurred and may notify the user through a user accessing the central server 100 or as above via automatic messaging. Central server 100 may have an easy-to-navigate website design with graphical user interface. Additionally, central server 100 may provide users the opportunity to enter additional health information that may be correlated against reported test results. Such health information may include stressful life events, eating habits, and exercise habits. Specific information concerning such occurrences may be correlated against testing results and provide significant additional information to medical personnel as to what types of lifestyle adjustments may greatly benefit particular patients.

Similar to above, medical personnel and doctors may receive notifications 140 via email, text, FAX, automated call, etc., when new test results or anomalous test results are posted. Anomalous test results may be those outside a particular range or those that show a particular percentage change.

Additionally, medical personnel, using portal 150, may receive access to central server 100 in order to review trends, add testing regimens for the patient, and adjust information that the user receives when the user logs into central server 100. Based on doctor assigned measurement schedules, test strips or other medical testing devices may be automatically sent to the patient. Doctors additionally may review trend and treatment progress across all of the doctor's patients, so a doctor may change his or her commonly prescribed treatment regimens. Additionally, the central server 100 may automatically provide information concerning treatment schemes to the test strip or medical detection device producers, so that the producers may better service doctors and patients by providing complementary product offerings and supply levels.

In many embodiments, parts of the system are provided in devices including microprocessors. Various embodiments of systems and methods described herein may be implemented fully or partially in software and/or firmware. This software and/or firmware may take the form of instructions contained in or on a non-transitory computer-readable storage medium. Those instructions then may be read and executed by one or more processors to enable performance of the operations described herein. The instructions may be in any suitable form such as, but not limited to, source code, compiled code, interpreted code, executable code, static code, dynamic code, and the like. Such a computer-readable medium may include any tangible non-transitory medium for storing information in a form readable by one or more computers such as, but not limited to, read only memory (ROM); random access memory (RAM); magnetic disk storage media; optical storage media; a flash memory; etc.

Embodiments of systems and methods described herein may be implemented in a variety of systems including, but not limited to, smartphones, tablets, laptops, and combinations of computing devices and cloud computing resources. For instance, portions of the operations may occur in one device, and other operations may occur at a remote location, such as a remote server or servers. For instance, the collection of the data may occur at a smartphone, and the data analysis may occur at a server or in a cloud computing resource. Any single computing device or combination of computing devices may execute the methods described.

While specific embodiments have been described in detail in the foregoing detailed description and illustrated in the accompanying drawing, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof. Note that, although particular embodiments are shown, features of each attachment may be interchanged between embodiments.

What is claimed as new and desired to be protected by Letters Patent of the United States is:

1. A system for measuring and reporting a level of an analyte, the system comprising:
a test strip having test strip layers configured to receive a blood sample from a user, and produce a detectable change in the test strip; and
an analyzer configured to read the detectable change in the test strip, the analyzer including an SMS message transmitter, the analyzer including a microprocessor including and executing software for converting a property read from the test strip into an analyte level and communicating that analyte level via SMS to a remote server.

2. The system of claim 1, wherein the analyzer is an optical analyzer.

3. A system for measuring and reporting a level of an analyte, the system comprising:
a test strip having test strip layers configured to receive a blood sample from a user, and produce a detectable change in the test strip;
an analyzer configured to read the detectable change in the test strip, the analyzer including an SMS message transmitter, the analyzer including a microprocessor including and executing software for converting a property read from the test strip into an analyte level and communicating that analyte level via SMS; and
a remote server including and executing software for receiving an SMS message including the analyte level.

4. The system of claim 3, wherein the analyzer is an optical analyzer.

5. The system of claim 3, wherein the remote server includes an SMS gateway.

6. The system of claim 3, wherein the remote server includes and executes software for storing the analyte level and correlating an indicator associated with the SMS message using a lookup table to determine the user associated with the analyte level.

7. The system of claim 3, wherein the remote server automatically sends an electronic message to the user when the user is to test for an analyte.

8. The system of claim 3, wherein the central server automatically sends a message to medical personnel when the analyzer reports an analyte level to the central server.

9. The system of claim 3, wherein the remote server provides a portal for the user to review historical test results.

10. The system of claim 9, wherein the remote server provides targeted information to the user through the portal.

11. The system of claim 10, wherein the targeted information includes advertisements for drugs based on the user's analyte level reported from the analyzer.

12. The system of claim 10, wherein the targeted information includes health management information based on the user's analyte level reported from the analyzer.

13. The system of claim 10, wherein medical personnel have access to modify the targeted information received by the user.

14. The system of claim 3, wherein medical personnel have access to modify the testing regimen executed by the user, and the remote server changes its notification schedule to the user accordingly.

15. The system of claim 14, wherein when medical personnel change the testing regimen for the user, testing supplies are automatically ordered for the user.

16. The system of claim 3, wherein the analyte level is automatically correlated against previous analyte levels by the remote server, and an indication is provided to the user if the analyte level is anomalous.

17. The system of claim 3, wherein the analyte level is automatically correlated against previous analyte levels by the remote server, and an indication is provided to medical personnel if the analyte level is anomalous.

18. The system of claim 16, wherein the indication is an SMS message.

19. The system of claim 17, wherein the indication is an SMS message.

20. The system of claim 16, wherein the indication is an email.
